(19) United States
(12) Patent Application Publication
(10) Pub. No.: US 2026/0248441 A1
(43) Pub. Date: Aug. 27, 2026

(54) METHOD AND DEVICE FOR ASSESSMENT OF PERIPHERAL NEUROPATHY

(71) Applicant: Otto-von-Guericke-Universitaet Magdeburg, Magdeburg (DE)

(72) Inventors: Peter R. Mertens, Magdeburg (DE); Antao Ming, Magdeburg (DE)

(21) Appl. No.: 18/870,508

(22) PCT Filed: May 31, 2023
(86) PCT No.: PCT/EP2023/064534

§ 371 Date: Nov. 29, 2024

(30) Foreign Application Priority Data
Jun. 1, 2022 (EP) 22176811.2

(51) Int. Cl. A61B 5/00; A61B 5/103

(52) U.S. Cl. CPC A61B 5/4041; A61B 5/1036; A61B 5/7271; A61B 5/743

(57) ABSTRACT

The invention relates to ways for determining, whether or not a person suffers from peripheral neuropathy. In order to make the generation of data for the determination easy for the person, the invention provides that sensor data from foot bottom pressure sensors (11, 12) used by the person to control computer games is used to determine, whether or not a person suffers from peripheral neuropathy.

### FIELD OF THE INVENTION

The invention generally relates to the assessment of peripheral nerve function of a person. In particular, the invention concerns a computer implemented method, a data processing device, a computer program and a system for indicating or determining, whether or not a person suffers from peripheral neuropathy.

### TECHNOLOGICAL BACKGROUND

From US 2012/0108909 A1 and US 2020/0129109 A1, it is known to assess brain injuries, bio-mechanical problems or other neurological disorders of the central nervous system using video games.

Impaired nerve function like peripheral neuropathy can lead to severe health issues which may lead to infection and may even require extremity amputation, in particular when the lower extremities are concerned. Yet, four out of five amputations may be prevented by adequate care and in case the impairment of the nerve function like peripheral neuropathy, particularly in the lower extremities, can be diagnosed in an early stage.

Impaired nerve function, in particular peripheral neuropathy, may be caused by diabetes. Currently, according to the International Diabetes Federation, more than 435 million people worldwide are diagnosed with diabetes. This number is expected to rise to 693 million in 2045. However, diabetes may cause impaired nerve function and in particular peripheral neuropathy even in a pre diabetes stage, which is often not diagnosed prior to the diabetes presenting itself with symptoms. When diabetes symptoms are present, however, the impairment of the nerve function may already have led to further health issues.

Symptoms of peripheral neuropathy may commonly originate distally (e.g. at the tips of the toes) and spread proximally with a symmetric distribution. Peripheral nerves encompass Aδ- and C-fibers (small, spinothalamic; temperature sensation, nociception, generally assessed by pinprick), as well as Aβ-fibers (large, back nervous system; generally assessed by vibration and monofilament). In addition, the reflex status may be altered (Achilles tendon reflex impairment).

Known ways to indicate or determine, whether or not a person suffers from peripheral neuropathy, either require a medical professional to perform tests and expensive tools or lab tests, or are insufficiently sensitive overall or with respect to differently sized peripheral nerve fibers whose function may be impaired.

### DESCRIPTION OF THE INVENTION

In view of the above-mentioned disadvantages, an object of the invention may be to provide a way to indicate or determine, whether or not a person suffers from peripheral neuropathy, in an accurate, enjoyable, time-saving, yet cheap manner, which allows for the patient to perform the examination without having a medical professional performing the examination and without the need to use expensive tools or lab tests.

This object is achieved according to the invention in that the above-identified computer implemented method for indicating or determining, whether or not a person suffers from peripheral neuropathy, comprises loading sensor data from foot bottom pressure sensors that were pressed between the feet of the person and the underground while the person was playing a predetermined computer game while controlling the computer game by exerting pressure on the pressure sensors via the person's feet, and analyzing the sensor data with an algorithm that is adapted to detect whether or not the person suffers from peripheral neuropathy based at least on the sensor data.

For the above-identified data processing device with a storage device and a processor, the object may be achieved in that in the storage device comprises instructions, which, when executed by the processor cause the processor to carry out the computer implemented method for indicating or determining, whether or not a person suffers from peripheral neuropathy according to the invention.

For the above-identified computer program comprising instructions, the object may be achieved in that the instruction, when the program is executed by a computer, cause the computer to carry out the computer implemented method for indicating or determining, whether or not a person suffers from peripheral neuropathy according to the invention.

For the above-identified system, the object may be achieved in that the system comprises a gaming device for playing computer games, at least two foot bottom pressure sensors that are connectable to the gaming device in a control signal transmitting manner, and the data processing device according to the invention, wherein the data processing device is adapted to load the sensor data from foot bottom pressure sensors, from the gaming device or from another storage device of the system.

By indicating or determining, whether or not a person suffers from peripheral neuropathy, based on data from foot bottom pressure sensors used for controlling a game, the person can easily and even remotely generate the examination data, which allows for the indication or determination of possible peripheral neuropathy. No supervision of the person while playing the game is required. Pressure sensors per se and particularly when compared to known examination equipment are low cost.

The solution according to the invention can be further improved by the embodiments mentioned in the following, which, unless explicitly mentioned to the contrary, can be combined as desired. The embodiments and their possible advantages are elaborated below:

According to an embodiment, the sensor data and game data concerning the person's gaming performance are combined and analyzed by the algorithm.

An advantage of this embodiment may be that the impairment of the peripheral nerve function can be indicated or determined with higher accuracy, i.e. higher sensitivity and/or higher specificity.

According to an embodiment, the sensor data include data representing the amount of pressure exerted by the person and/or the timing of the pressure exerted by the person, optionally combined with game control timing achieved by the person, possibly in comparison to at least one preset threshold value for pressure amount and/or pressure timing.

An advantage of this embodiment may be that using data that represents different aspects of the pressure exerted by the person may further improve accuracy, i.e. sensitivity and/or specificity.

According to an embodiment, the game data include timing and/or accuracy of control of positioning an item of the game with respect to another item of the game.

An advantage of this embodiment may be that using data that represents different aspects of gaming performance of the person may further improve accuracy, i.e. sensitivity and/or specificity.

For example, data derived from the sensor data and/or the game data may be reaction time, anticipation time, time/frequency inside/outside of a predetermined optimal area, deviation to a predetermined ideal pressure/position, normalized pressure difference, normalized pressure gradient, normalized pressure time integral, overall sensation, skillfulness, muscle strength of lower limbs, plantar pressure deviation and/or endurance.

Particularly, if the sensor data and the game date is combined according to an embodiment, the accuracy of the indication or determination may be improved.

According to an embodiment, the algorithm is adapted to derive the person's capabilities from the sensor and optionally the game data, wherein the algorithm may be adapted to indicate or determine the person's reaction time, skillfulness, muscle strength (lower limbs), plantar pressure deviation and/or endurance as the person's capabilities from the sensor data or the combination of sensor data and game data.

An advantage of this embodiment may be that derived not only raw data, but also derived capabilities of the person using the sensors to control the game can be used, which may further improve the accuracy of the indication or determination.

According to an embodiment, the algorithm is an artificial intelligence model trained to indicate or determine whether a person suffered from impaired peripheral nerve function, in particular peripheral neuropathy, based at least on the pressure data.

An advantage of this embodiment may be that parameters that were not thought to be indicative for peripheral neuropathy can be found during training of the artificial intelligence model and used by the trained artificial intelligence model. Alternatively, or additionally, the using the trained artificial intelligence model may further improve the accuracy of the indication or determination.

According to an embodiment, the computer game is a game of skill.

An advantage of this embodiment may be that games of skill not only provide a high motivation for playing, but further support by provoking the person who is playing the game to perform comparatively large and comparatively small movements with high and low forces, such that different fibers and reflexes are applied while playing the game of skill, which facilitates indication or determination of impaired function of differently sized fibers.

According to an embodiment, the system comprises shoe inlays or shoes, wherein the inlays of the inner sole of the shows each comprise at least one of the foot bottom pressure sensors. Alternatively, the foot bottom pressure sensors may be affixed to the person's foot bottoms, wherein this embodiment could require support by a third person for affixing the sensors. As a further alternative, the foot bottom pressure sensors may be provided in or at parts of socks that contact the foot bottoms of the person when the person is wearing the socks.

For example, pair of shoes harboring sensor-equipped insoles available by AntiSense System, IEE S.A. Luxembourg, may be used. These insoles each encompass an Electronic Control Unit (ECU) and e.g. eight pressure sensors (5.57 cm² high dynamic HD002 force sensing resistors) that may be integrated into the heel, lateral arch, metatarsal 1, 3, and 5, hallux, and toe region of the foot. The sensors may allow for pressure detection with a sensitivity of 3.4 mbar in the range between 250 mbar and 7 bar. The ECU may comprise a nine degrees of freedom inertial measuring unit (IMU, embedding a 3-axis accelerometer, a 3-axis gyroscope, and a 3-axis magnetometer), sampling rate up to 500 Hz, data synchronization (between insoles and smart devices), automatic detection of foot side, internal storage of 16 Gigabytes, and up to 10 h energy supply. The sensors can be embedded in foil and may not protrude. The insole can be integrated into footwear with a support layer of ethylene-vinylacetat-30 material and a protective layer of sponge.

Sensor data can be recorded at more than 50 Hz, more than 100 Hz, up to or more than 200 Hz and transferred in real-time to the gaming device e.g. via Bluetooth or via cable for smooth steering of games. The person using the system for indicating or determining, whether or not the person suffers from peripheral neuropathy, may be seated on a chair without armrests in front of a table on which the gaming device, e.g. a tablet computer (for example Samsung Galaxy Tab A T580 or an iPad) can be positioned that connects to the insole via Bluetooth 5.0 or cable. The persons may initially calibrate the insole through eight predefined steps.

An advantage of this embodiment may be that no hardware needs to be specifically developed, but already available hardware can be used, which further reduces cost and increases availability.

For example, pressure thresholds measured during a calibration a person conducts with the foot bottom pressure sensors, can be used to normalize the absolute pressure values to the range of zero to one. Subsequently, the person can familiarize him- or herself with the setup and control techniques of the video games through standardized tutorials that can be repeated on demand. Each gaming session may consist of for example at least one of or up to all of the following four games: Apple-Catch (AC), Balloon-Flying (BF), Cross-Pressure (CP), and Island-Jump (IJ), which can be played sequentially. Each game session may last at least 5 minutes, up to 10 minutes, up to 15 minutes or up to 20 minutes. The acquired sensor data and game outcomes can be transferred in a complete data package to a remote server for data visualization and analyses or can be transferred to another computer for data visualization and analyses or can be analyzed and/or visualized by the gaming device the game is played on. A spider chart can provide feedback on the game performance to the person after the session (exemplary key capabilities: reaction time, sensation, skillfulness, muscle strength (lower limbs), plantar pressure deviation and/or endurance).

A data processing device and/or a gaming device may be a computer with a processor, with a solid, non-transitory storage, with RAM, data interfaces like USB, HDMI, DisplayPort etc., and may in particular be any Personal Computer, desktop computer, laptop computer, tablet computer, smart phone, smart watch, server etc.

Therefore, in other words, peripheral neuropathy (PNP) is a common comorbidity in patients with diabetes or metabolic syndrome (MetS) that is diagnosed by clinical examination (e.g., pinprick, vibration perception, Tip Therm, monofilament, reflexes). These are time-consuming and rarely performed in primary care centers by caring physicians, likely due to time restrictions.

Early recognition of PNP is a hallmark to prevent complications, e.g., foot ulceration. Clinical examination includes standardized procedures (pinprick, vibration perception, Tip Therm, monofilament, reflexes). These analyses are subject to investigator-related bias and are performed with low frequency in primary practice, likely due to time restrictions.

PNP is a challenge to diagnose for primary care clinicians due to its diverse forms and presentations. Especially in patients with diabetes mellitus or MetS, the onset is insidious and most affected individuals are not aware of distal symmetric PNP. Further common causes consist of excessive and chronic alcohol use, nerve injury, toxin exposure, nutritional deficiencies, chemotherapy, and genetic disorders.

According to the International Diabetes Federation (IDF), more than 435 million people worldwide are diagnosed with diabetes, which is expected to rise to 693 million in 2045. 25% to 50% of patients with diabetes are impacted by PNP, and the severity varies according to age, duration of diabetes, and level of glucose control. PNP significantly impacts the mobility of patients, associated with disturbed gait and coordination, increased likelihood of falls, higher incidence rates of diabetic foot syndrome, and frail mental health. Besides impaired sensation, PNP may also cause plus symptoms of discomfort and pain. Complications such as tissue damage, infections, and ultimately minor and major foot ulcerations are estimated to affect 19% to 34% of individuals with diabetes during their lifetime, especially with delayed diagnosis of PNP or inadequate preventive measures. Notably, every fifth moderate-to-severe diabetes-related infection will prompt lower extremity amputation. On the other hand, four out of five amputations may be prevented by adequate podiatry care.

All these aspects urge for the optimization of PNP diagnosis, possibly even prevention and management. Due to the nature of the disease, timely diagnosis and repeated monitoring of individuals at risk are mandatory, given the insidious onset of PNP with diverse clinical presentation. Up to 50% of affected individuals remain asymptomatic, while the remainder develop numbness, tingling, pain, or weakness. Symptoms commonly originate distally (i.e., at the tips of the toes) and spread proximally with a symmetric distribution. Peripheral nerves encompass large nerve fibers (Aα- and Aβ-fibers; vibratory and pressure sensation) and small nerve fibers (unmyelinated C- and Aδ-fibers; pain and warm/cold sensation).

Due to their invasive nature, nerve conduction studies (NCS) and skin biopsies are not applicable as screening tools. According to the position statement published already 15 years ago by the American Diabetes Association (ADA), “assessment should include a careful history and either temperature or pinprick sensation [. . . ] and vibration sensation [. . . ]”. Some innovations as point-of-care devices have been brought forward, however, are not established as routines. Since PNP diagnosis is regarded as a game changer for intervention and thus prevention of complications, innovative strategies are highly needed.

According to the invention, a game-based examination platform consisting of four video games guided by pressure sensor-equipped insoles as control units is provided. In patients diagnosed with diabetes or MetS, game performance is correlated with clinical PNP findings by neuropathy symptom scores, neuropathy disability scores and NCS.

To establish and evaluate a playful nerve function assessment device through video games that are coordinated by foot movements with foot bottom pressure sensors, e.g. pressure sensor-equipped insoles. Pressure recordings may be transferred in real-time to a central database for analyses. Games can be designed with different challenges; feature extraction methodologies can be applied and tested in a pilot exploratory study. The game-based approach was evaluated in terms of correlation with NCS and clinical findings (NDS, NSS) by predefined hypothesis-driven parameters (reaction time, sensation, skillfulness, muscle strength (lower limbs), plantar pressure deviation and endurance). Exemplary representative game features showing significant differences between patients with small fiber or large fiber peripheral neuropathy (SFN, LFN) were identified. In addition, exemplary training and optimization of classification models for PNP and clinical phenotypes were reported using selected game features.

The game-based approach allows to identify patients with PNP based on their gaming performance. Their task was to perform a parcours of digital games that are guided through pressure sensor-equipped insoles. When correlated with clinical neuropathy scores and nerve conduction studies, the execution of games with the developed algorithms yielded accuracies of 76.1% and 81.7% for the left and right foot, respectively. In addition, some features of the gaming session allowed to subclassify the PNP damage pattern (small, large, or mixed nerve fiber dysfunction).

For example, out of 329 persons, clinically evident PNP was diagnosed in 247 (75.1%). Asymmetric findings were detected in 88 (26.7%) individuals. In a subcohort undergoing NCS as the gold standard, highly significant correlations were found for game performance with sensory and motor nerve conduction velocity (NCV) as well as major nerve amplitude in lower extremities (R=0.65, p<0.001, adjusted R2=0.36). In an age-matched subcohort 2, significant correlations were determined for the severity of PNP and hypothesis-driven key capabilities for game performance. Learning models derived with extracted game features achieved accuracies of 76.1% (left) and 81.7% (right foot) for the detection of PNP. The obtained multi-classification models yielded a multi-class AUC of 0.76 (left) and 0.72 (right foot) when assessing the PNP damage pattern (small, large, or mixed nerve fiber damage).

Thus, a game-based neuropathy evaluation platform yields a meaningful assessment of PNP and qualifies for examiner-independent testing in primary care.

The inventors found that the design of standardized video-based games allows to derive important information on the neuropathy status of patients. With the foot bottom pressure sensors, e.g. pressure-sensor-equipped insoles, an overall complex interplay of game perception and execution in a standardized environment is achieved that yields data sets from sensor data for sample analyses. The chosen approach allows to enter these data for classification into subgroups of patients with different degrees of PNP and affection of small/large nerve fibers. Thus, screening of patients for PNP is feasible in a playful manner, even as a telemedical application. Thus, in an experimental setup, it was feasible at remote, and provided a simplified tool that may potentially be easily adopted in primary care.

For the development of an exemplary suitable artificial intelligence (AI) algorithm, binary classification models were derived to identify feet with PNP within individuals diagnosed with diabetes or metabolic syndrome using game parameters extracted from acquired data sets.

For example, for AI modeling, acquired data sets were initially split into testing and training data sets (ratio 3:7). The random sampling occurred in each class and preserved the overall class distribution of the data. Only the training data set was utilized to identify features with significant between-group differences and imported to the classifier for estimating the importance of features depending on model-independent metrics. Subsequently, models with different subsets of top-ranked features (i.e., variances of numbers and/or orders of features) were tested. Multiple classifiers including Random Forest (RF), Lasso and Elastic-Net Regularized Generalized Linear Models (GLMNET), Support Vector Machines (SVM), and Gradient Gradient Boosting (GBM) were tested during modeling. The area under the receiver-operating-characteristic curve (AUC-ROC) was selected as the performance metric to compare the models trained with different feature combinations. The five-fold ten repeats cross-validation was utilized in training to avoid overfitting and derive a more accurate estimate of the model performance. This statistical method repeatedly divided the training data set into five subsets with approximately equal size three times. Each subset contained the same proportion of labels as the complete data set. Four of the five subsets were utilized in the model training, while the remaining subset was used for validation. The average AUC of cross-validation was considered in the grid search of parameters combination that improves the model performance the most. Ultimately, the obtained models were further applied to the hold-out testing data set to evaluate the models' predictive performance. Classification performance was evaluated by calculating AUC-ROC, balanced accuracy, sensitivity, and specificity, as compared with a reference standard of clinical examination.

Besides, multiple classification models were trained to predict small, large, or mixed nerve fiber damage of the feet in patients with diabetes or metabolic syndrome. All data sets were utilized for modeling and the five-fold ten repeats cross-validation was applied due to limited sample size and imbalanced class distribution. The multi-class AUC was computed to evaluate the model performance. R programming language (version 4.2.1) and related open-source libraries were utilized for statistical computing and machine learning algorithms.

Game features were analyzed during the development phase as follows: NCS was performed in 37 patients (subcohort 1), all diagnosed with diabetes and normal cognitive status (MoCA≥26). This subcohort has similar distributions in age, weight, BMI, and neuropathic scores as the entire cohort. The gender distribution is lightly skewed towards female patients in subcohort 1. Patients' game performance was significantly correlated to the sensory and motor conduction velocity (NCV) and amplitude in the lower extremities. Feature extraction of distinct parameters from four games was set up for each foot side. Overall, 277 independent game features revealed obvious associations with NCS parameters. A correlation was observed between the motor nerve conduction amplitude of the right foot and the maximal execution time in task 8 of the Island-Jump game (R=0.65, p<0.001, adjusted R2=0.36). BF and AC games contributed more features that were correlated with the NCV and amplitude of the left foot. However, more features extracted from BF and IJ games were associated with the NCV and amplitude of the right foot. 164 and 113 independent game features were significantly correlated with motor and sensory nerve states, respectively. 146 independent game features were found to be associated with NCV, and 131 were correlated to the amplitude of nerves of the foot.

Out of all persons (n=329), a subcohort 2 with normal cognitive state (MoCA≥26) aged >55 years was generated. No significant differences in age, gender, and BMI were observed between patients without and with clinical PNP. Individuals with neuropathy had a significantly longer duration of diabetes and more severe neuropathic symptoms. Clinically evidence for PNP was present in 118 left and 120 right feet. For all these feet, at least one insensate site during the 10-g monofilament test were found. PNP was identified in both feet in 112 patients. No PNP was present in 47 patients, 26% of patients had unilateral PNP.

Critical skills related to nerve functions are required for successful game performance. As an example, four hypothesis-driven key capabilities were defined: reaction time (understanding; immediate response to tasks), sensation (fine-tuning of pressure application in subtasks), skillfulness (overall achievements in each game), muscle strength of lower limbs (achievements with high-pressure application), plantar pressure deviation (pressure distribution left versus right foot) and endurance (steadiness of pressure application in tasks). All key capabilities revealed significant differences between patients without PNP (n=47) and with symmetrical PNP (n=112).

With the extracted game features from 173 patients in subcohort 2, predictive models for PNP were trained exemplarily using the five-fold ten repeats cross-validation. For example, 70% of game data sets were utilized for model training and 30% for testing. In the hold-out testing data set, the obtained Lasso and Elastic-Net Regularized Generalized Linear Model (GLMNET) identified PNP in the left foot with an adjusted accuracy of 76.1% (sensitivity 77.1%, specificity 75.0%, AUC-ROC 0.72). For determining PNP in the right foot, another GLMNET model achieved an accuracy of 81.7% (sensitivity 83.3%, specificity 80.0%, AUC-ROC 0.75).

In addition, for example, multi-classification models were established to differentiate feet without PNP (w/o) versus SFN, LFN, and mixed nerve fiber damages. Exemplary game features were extracted separately for the left and right foot from the game data set. The trained Gradient Boosting Models (GBM) yielded a multi-class AUC of 0.76 and 0.72 when identifying the nerve fiber damage pattern for the left and right foot, respectively. Both models performed better in distinguishing between SFN and LFN.

Thus, in summary, a game-based setup to detect PNP in patients with diabetes mellitus or MetS is feasible and accurate. Games can be designed to detect key capabilities related to nerve functions with result interpretations based on hypothesis-driven assumptions as well as algorithms developed by Al methodologies. The findings from NCS data suggest that a standardized gaming session of a duration between 5 and 25 minutes and for example 15 minutes length is sufficient to acquire a rather objective assessment of peripheral nerve function. The extracted game features are associated with both, NCV and amplitude of the major nerves of the foot, suggesting a possible combination of axonal degeneration and segmental demyelination. Learning models trained with gaming data sets from an age-matched subcohort allowed for the discrimination between feet with PNP and healthy feet with accuracies of 76.1% and 81.7% for each foot side, respectively. The definition of PNP is based on the widely used diagnostic criteria (a combination of NSS/NDS) but implemented separately for each foot side. Clinically evident PNP was identified in 75.1% of study persons, suggesting a very high risk of developing foot complications in the cohort. The chosen diagnostic criteria according to clinical examination allowed to discriminate between nerve damage patterns of PNP. The obtained multi-classification models predicted the nerve damage pattern (small, large, or mixed fiber neuropathy) with AUC of 0.76 and 0.72 for the left and right foot, respectively.

Asymptomatic neuropathy was observed in 30% of the study persons. This underscores the importance of developing such easy-to-use screening tools to avoid or delay the progression of PNP. In addition, more than one-fourth of persons were diagnosed with asymmetric neuropathy. To circumvent the methodological errors caused by combining analyses with asymmetric neuropathy, analyses and modeling can be performed separately for each foot.

The game-based approach may be performed highly standardized, largely investigator-independent, and does not require health care professionals. The barrier-to-completion in this study is low, even for those with little or no gaming experience. The immediate response of the studied persons upon completion of the games was overwhelmingly positive. More than 90% wished to repeat the sessions. However, the results of the second course were not included in the study results reported in this disclosure. As a visionary outlook of a telemedicine setting, individuals may perform game sessions at home with the distribution of results to caring physicians remotely following predictions of the AI models. This approach may be amenable for high-risk individuals in primary care settings with seniors suffering from limited mobility or geographic restrictions.

In this disclosure, the following abbreviations may have been used:

AC: Apple-Catch; ADA: American Diabetes Association; AUC-ROC: Area under the Receiver Operating Characteristic Curve; BF: Balloon-Flying; CP: Cross-Pressure; PNP: Peripheral Neuropathy; ECU: Electronic Control Unit; GBM: Gradient Boosting Models; GLMNET: Lasso and Elastic-Net Regularized Generalized Linear Models; IDF: International Diabetes Federation; IMU: Inertial Measuring Unit; IJ: Island-Jump; LFN: Large Fiber Neuropathy; NDS: Neuropathy Disability Score; NSS: Neuropathy Symptom Score; NYHA: New York Heart Association; NCS: Nerve Conduction Studies; PNP: Peripheral Neuropathy; SD: Standard Deviation; SFN: Small Fiber Neuropathy; SVM: Support Vector Machine; TCL: Task Combination for Left Foot; TCR: Task Combination for Right Foot.

### BRIEF DESCRIPTION OF THE DRAWINGS

The invention is described hereinafter in more detail and in an exemplary manner using advantageous embodiments and with reference to the drawings. The described embodiments are only possible configurations in which, however, the individual features as describes above can be provided independent of one another or can be omitted in the drawings:

For elements of an exemplary embodiment, which correspond in form and/or function to elements of another exemplary embodiment, the same reference numerals are used.

FIG. 1 shows an exemplary embodiment of a computer implemented method for indicating or determining, whether or not a person suffers from peripheral neuropathy, with steps that comprise gathering sensor data, which are not part of the claimed invention,

FIG. 2 shows an exemplary embodiment of a system for indicating or determining, whether or not a person suffers from peripheral neuropathy,

FIGS. 3 to 7 show exemplary embodiments of games played by the person by means of foot bottom pressure sensors,

FIG. 8 shows an exemplary embodiment of a classification modeling scheme of an artificial intelligence,

FIG. 9 schematically shows functionalities of an exemplary embodiment of the computer method implemented as the artificial intelligence,

FIG. 10 schematically shows an overview of an exemplary embodiment of the system for indicating or determining, whether or not a person suffers from peripheral neuropathy,

FIG. 11 schematically shows performance in nerve conduction study and correlation with game-based findings,

FIG. 12 schematically shows a possible classification of peripheral neuropathy based on game-generated sensor data,

FIG. 13 schematically shows another possible classification of peripheral neuropathy based on game-generated sensor data,

FIG. 14 schematically shows a calibration routine for calibrating the foot pressure sensors,

FIG. 15 schematically shows extraction and analysis of features from game-generated sensor data for a peripheral neuropathy classification model and

FIG. 16 schematically shows considerations on key capabilities and scoring of game performance and game-generated sensor data.

### DESCRIPTION OF EXEMPLARY EMBODIMENTS

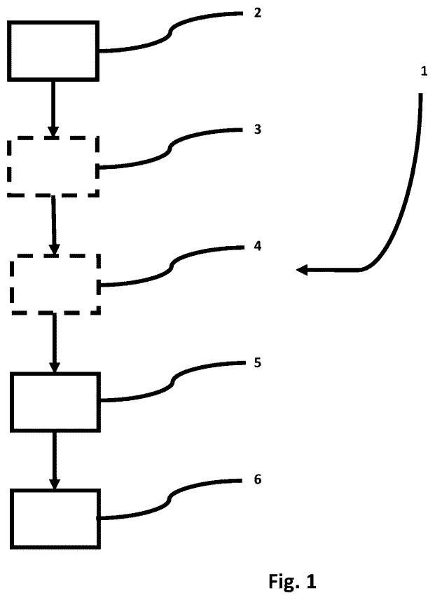

FIG. 1 shows a first exemplary embodiment of a computer implemented method for indicating or determining, whether or not a person suffers from peripheral neuropathy, according to the invention and as a flow chart.

The computer implemented method 1 for indicating or determining, whether or not a person suffers from peripheral neuropathy, starts with a first method step 1. Method steps 3 and 4 concern bringing at least one foot bottom pressure sensor in contact with each of the person's feet (step 3) and to let the person play the games while gathering pressure and optional game data (step 4). Method steps 3 and 4 are not part of the claimed invention, but are useful for the understanding of the invention as claimed. Steps 3 and 4 may be supervised by another person, which may even assist the person that may have peripheral neuropathy, wherein the other person needs not to be of any medical profession.

In method step 5, the sensor data and optionally also the game data is analyzed with an algorithm that is adapted to detect whether or not the person suffers from peripheral neuropathy based on the sensor data.

Finally, the method 1 may end with method step 6, in which the results of the indication or determination may be communicated with the person or with another person, e.g. a medical practitioner usually examining or treating the person, like a general or specialized practitioner. The other person needs not to be present for applying the sensors or while playing the games.

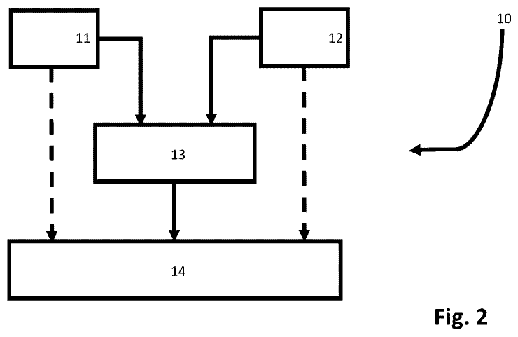

FIG. 2 schematically shows an exemplary embodiment of the system for indicating or determining, whether or not a person suffers from peripheral neuropathy, according to the invention.

The system 10 comprises at least two foot bottom pressure sensors 11, 12, each of which being applied to the bottom of another foot of the person, such that the person can control games by differently pressing her or his feet on the ground. The system 10 further comprises a gaming device 13 for playing computer games. The foot bottom pressure sensors 11, 12 are connectable to the gaming device 13 in a control signal transmitting manner. The control signal depends and for example may be proportional to the pressure the person applies to the ground vis the foot bottom pressure sensors 11, 12. The control signal may be converted into sensor data by the foot bottom pressure sensors 11, 12 or by the gaming device 13. Optionally, the foot bottom pressure sensors 11, 12 may be connected in a signal and e.g. data transmitting manner to a data processing device 14 of the system 10. The data processing device 14 may, hence, be adapted to load the sensor data from foot bottom pressure sensors 11, 12, from the gaming device 13 or from another storage device of the system 10. The data processing device 14 may be the gaming device 13 or a part thereof, may be provided separate from the gaming device 13 and may be connected directly, e.g. wirelessly or via a cable, or via the internet to the gaming device 13 and/or the foot bottom pressure sensors 11, 12.

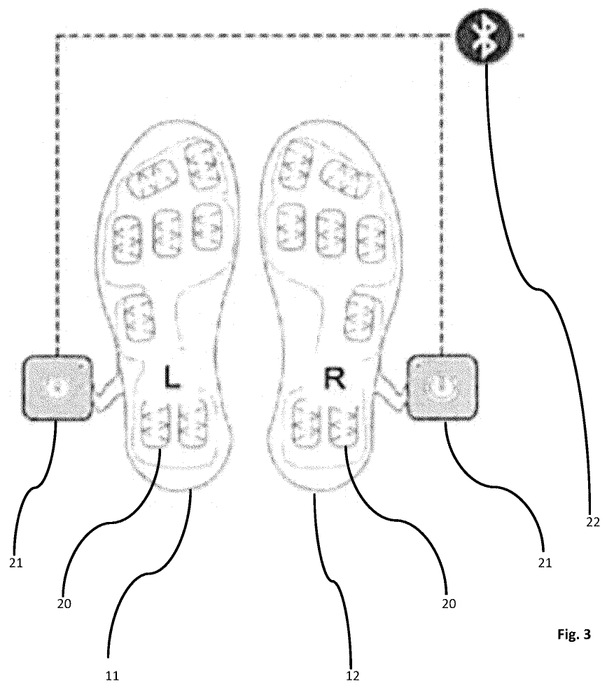

FIG. 3 shows an exemplary embodiment of foot bottom pressure sensors 11, 12 in a schematic view.

The foot bottom pressure sensors 11, 12 may each comprise at least one or more than one pressure sensitive element 20, i.e. pressure sensor element. The foot bottom pressure sensors 11, 12 may be shoe inserts. The foot bottom pressure sensors 11, 12 may comprise a common signal to data converter 21, which is connected to the pressure sensitive elements 20 and converts the sensor signal into sensor data. The signal to data converter 21 may be an analog to digital converter. The exemplary embodiment of FIG. 3 shows the two foot bottom pressure sensors 11, 12, wherein each of the foot bottom pressure sensors 11, 12 comprises a signal to data converter 21. The foot bottom pressure sensors 11, 12 may a signal or data interface 22 that is adapted to output the sensor signal or the sensor data. The signal or data interface 22 may be a physical interface like a connector. The exemplary embodiment of FIG. 3 shows the two foot bottom pressure sensors 11, 12 with a wireless signal or data interface 22, which may output the sensor data wirelessly, e.g. via Bluetooth.

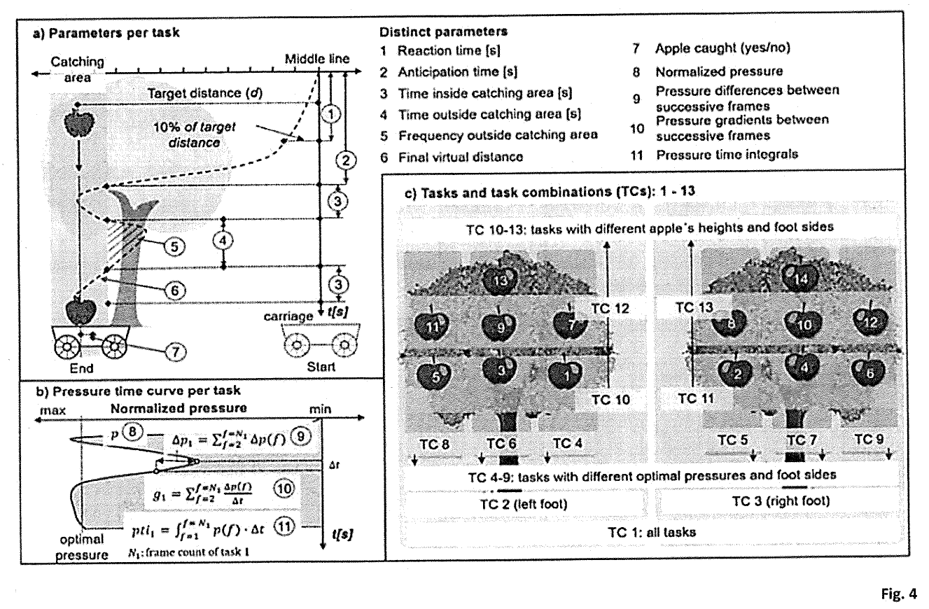

FIGS. 4 to 7 schematically depict games and how to control these games to generate the data, which, according to the invention, is used for indicating or determining, whether or not a person suffers from peripheral neuropathy.

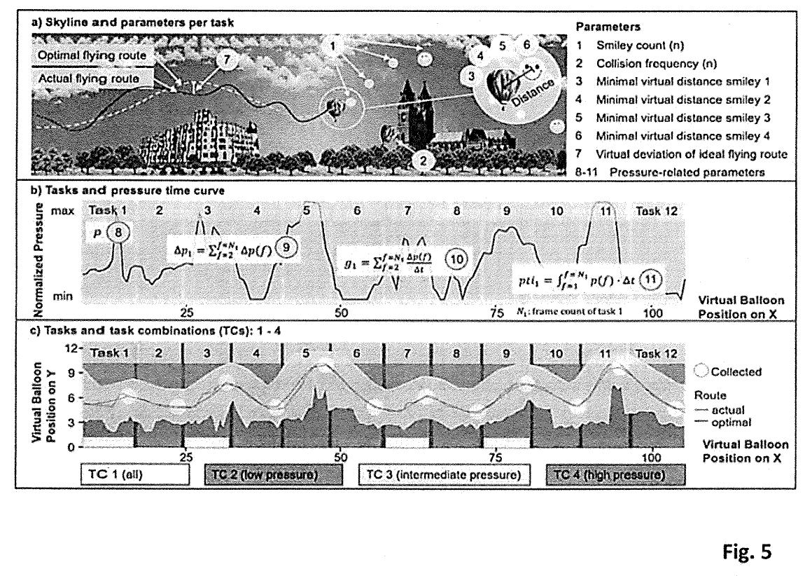

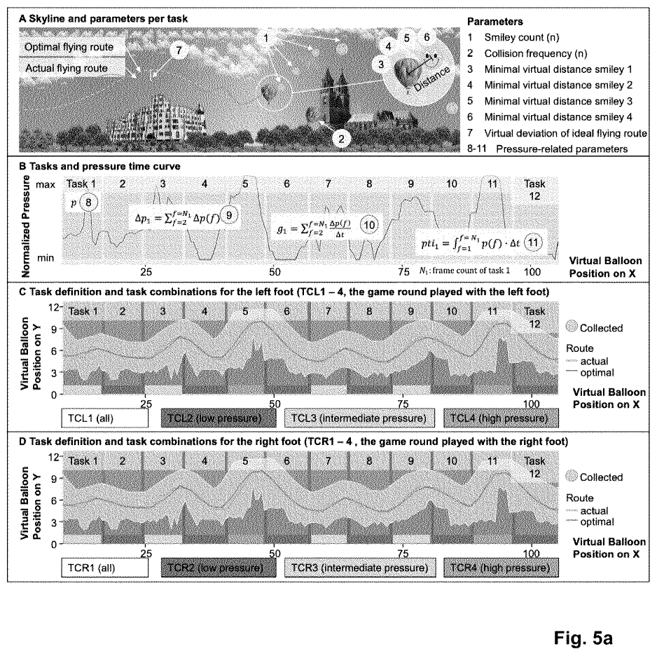

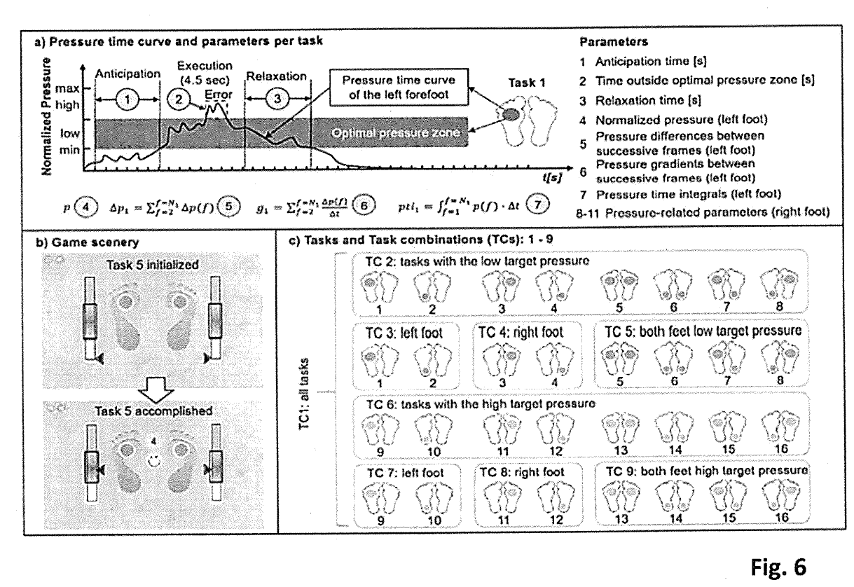

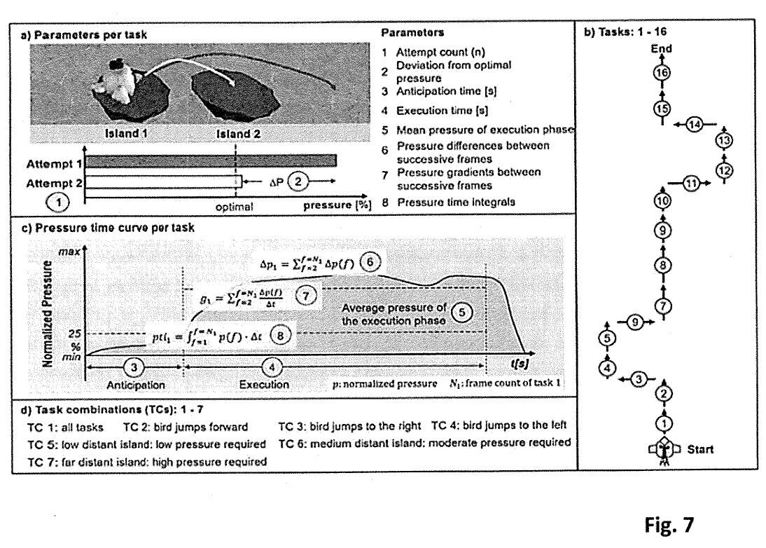

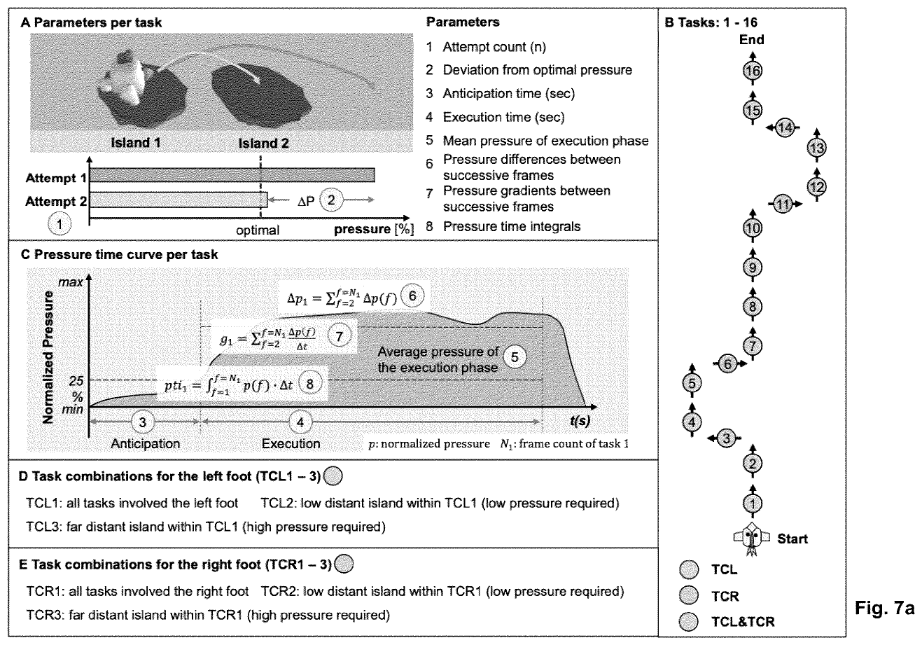

The measured pressure thresholds may be utilized to normalize the absolute pressure values, e.g. to the range of zero to one. Subsequently, the person may familiarize her-or himself with the setup and control techniques of the computer games through predefined tutorials that may be repeated on demand. Each gaming session may comprise a plurality of e.g. four games: Apple-Catch (AC, FIG. 4), Balloon-Flying (BF, FIG. 5), Cross-Pressure (CP FIG. 6), and Island-Jump (IJ, FIG. 7), which can be played selectively or sequentially. Each game may last several minutes, e.g. up to 5 minutes, up to 10 minutes, up to 15 minutes or about 20 minutes. The acquired sensor data and game data can transferred in a complete data package to a remote server for data visualization and analyses.

In the AC game, the person positions a carriage to catch apples falling from tree. The BF game challenged the person to fly a balloon along a predetermined path. The CP game required the person to provide predefined loads with predefined parts of the feet in predefined patterns. In the IJ game, the person controls an avatar to jump from one island to another island, wherein the at least one pressure variable like the amount of pressure of the timing of the pressure of the duration of the pressure determined whether the avatar lands on the other island. A known game with a similar approach but with a different control mechanism may be “Hot Lava” by Klei Entertainment Inc., which is controlled by game pads or tough screens, i.e. by hand. All movements of items (the carriage of the AC game, the balloon of the BF game, the avatar of the IJ game) and/or indicators on the CP game are controlled by the sensor signal, which depends from the person exerting pressure with his feet on the pressure sensors.

Game data extraction of distinct game parameters from defined game tasks and game task combinations for the games can be set up. Game parameters can be calculated from each game task and can be considered as primary features, such as reaction time e.g. in the first task of the AC game. Apart from that, the concept of task combination (TC) can be introduced, i.e., a set of game tasks with similar specifications or macro-measurements can be combined. For example, TC1 of the AC game can encompass all tasks of this game. TC2 can cover all tasks that can use the left foot for controlling the carriage movement to catch apples. The corresponding TC3 can consist of all tasks that use the right foot. The sum, mean, and standard deviation of primary features over game tasks of TCs can be treated as secondary features. For example, in the AC game, the reaction time of TC1 can be a secondary feature that can be computed from the average reaction time over all tasks. Overall, 1.880 distinctive parameters reflexing person's performance in the entirety and among similar tasks can be extracted per data set (per person and for all four games in total).

The games may provide sets of data that comprise the sensor data and optionally the game data, wherein the AC game may provide 583 data elements or features, the BF game may provide 528 data elements or features, the CJ game may provide 473 data elements or features, and the IJ game may provide 296 data elements or features, such that, in case the person plays all four games, in total 1880 data elements or features are present in the data set of sensor and optionally game data to indicate or determine, whether or not a person suffers from peripheral neuropathy.

Compared to FIG. 5, FIG. 5a, additionally shows a field D, in which the position data of the virtual balloon controlled with the right foot is depicted in comparison with optimal positions. Compared to FIG. 7, FIG. 7a, additionally shows a field E, in which right foot data of the island jump game is depicted.

FIG. 8 shows a classification scheme of an exemplary embodiment of an algorithm representing an AI (Artificial Intelligence) model for indicating or determining, whether or not a person suffers from peripheral neuropathy.

For example, AI (Artificial Intelligence) models can be derived to distinguish healthy controls and patients with diabetes and sensory neuropathy in an age-matched cohort (cohort 2) with acquired data sets. The NDS (neuropathy deficit score) can be utilized to generate class labels for supervised AI learning. NDS greater than or equal to 2 and impaired vibration sensation (0-4/8) can be considered as existing neuropathy (DPN), and NDS=0 can be considered as the absence of neuropathy (Control group). Secondly, in the DPN group, more detailed phenotyping can be achieved by analyzing the dysfunction of different fiber types. FIG. 8 summarizes the detailed information of these modeling schemes. For model 2 of FIG. 8, severe damage of Aδ/C-fibers can be assumed in patients with reduced/absent pinprick (nociception) or temperature sensation. For model 3 of FIG. 8, the presence of severe Aβ-fiber polyneuropathy can be assumed with impaired vibration sensation (below ⅜) or an abnormal 10 g monofilament test result (reduced/absent). The remaining patients in the DPN group can be classified as moderate Aβ-fiber polyneuropathy (impaired vibration sensation: 3- 4/8). For model 4 of FIG. 8, the absence of Achilles tendon reflexes can be considered as a positive label in the model training. A negative label may be assigned to normal or impaired reflexes.

For feature selection, acquired data sets can be initially split into testing and training data sets (e.g. ratio 3:7). The random sampling can occur in each class and preserved the overall class distribution of the data. Only the training data set can be imported to the classifier for estimating the importance of features depending on model-independent metrics, e.g., area under the receiver operating characteristic. Subsequently, models with different subsets of top-ranked features (i.e., variances of numbers and/or orders of features) can be tested. If the studied data set is imbalanced, Cohens Kappa (a classification accuracy normalized by the imbalance of the classes in the data) can be selected as the performance metric to compare the models trained with different feature combinations. The models with higher Cohens Kappa values can be chosen as candidate models,

Support vector machine (SVM) with three kernel functions (linear, radial, and polynomial) can be selected for model training. Clinical studies reported that SVM models perform better on medical classification issues with limited and imbalanced data sets. Three repeats five-fold cross-validation can be utilized in feature ranking and training to avoid overfitting and derive a more accurate estimate of the model performance. This statistical method can repeatedly divide the training dataset into e.g. five subsets with approximately equal size three times. Each subset can contain the same proportion of labels as the complete dataset. A subset and e.g. four of the five subsets can be utilized in the model training, while the remaining subset can be used for validation. The average accuracy of cross-validation can be considered in the grid search of parameters combination that improves the model performance the most. Ultimately, the obtained candidate models can be further applied to the testing data set to evaluate the models' predictive performance.

R programming language (version 4.0.4) and related open-source libraries can be utilized for statistical computing and machine learning algorithms.

For example, libraries that may be used are mentioned in the following (Package, Version, Description, Source, Published Date):
Caret, 6.0-86, misc functions for training and plotting classification and regression models, Comprehensive R Archive Network (CRAN), 2020-03-20 10:20:07 UTC
Colorspace, 2.0-0, “Carries out mapping between assorted color spaces including RGB, HSV, HLS, CIEXYZ, CIELUV, HCL (polar CIELUV), CIELAB, and polar CIELAB, qualitative, sequential, and diverging color palettes based on HCL colors are provided along with corresponding ggplot2 color scales, color palette choice is aided by an interactive app (with either a Tcl/Tk, or a shiny graphical user interface) and shiny apps with an HCL color picker and a color vision deficiency emulator. Plotting functions for displaying and assessing palettes include color swatches, visualizations of the HCL space, and trajectories in HCL and/or RGB spectrum. Color manipulation functions include: desaturation, lightening/darkening, mixing, and simulation of color vision deficiencies (deutanomaly, protanomaly, tritanomaly), Details can be found on the project web page at http://colorspace.R-Forge.R-project.org/ and in the accompanying scientific paper: Zeileis et al. (2020, Journal of Statistical Software, <doi: 10.18637/jss.v096.101>).”, Comprehensive R Archive Network (CRAN), 2020-11-11 07:40:15 UTC
Corrplot, 0.84, “A graphical display of a correlation matrix or general matrix. It also contains some algorithms to do matrix reordering. In addition, corrplot is good at details, including choosing color, text labels, color labels, layout, etc.”, Comprehensive R Archive Network (CRAN), 2017-10-16 22:58:08 UTC
Dfidx, 0.0-4, Provides extended data frames, with a special data frame column which contains two indexes, with potentially a nesting structure, Comprehensive R Archive Network (CRAN, 2021-02-03 05:40:10 UTC
Dplyr, 1.0.7, “A fast, consistent tool for working with data frame like objects, both in memory and out of memory.”, Comprehensive R Archive Network (CRAN), 2021-06-18 23:20:01 UTC
Egg, 0.4.5, Miscellaneous functions to help customise ‘ggplot2’ objects. High-level functions are provided to post-process ‘ggplot2’ layouts and allow alignment between plot panels, as well as setting panel sizes to fixed values. Other functions include a custom ‘geom’, and helper functions to enforce symmetric scales or add tags to facetted plots, Comprehensive R Archive Network (CRAN), 2019-07-13 06:00:27 UTC
Formula, 1.2-4, “Infrastructure for extended formulas with multiple parts on the right-hand side and/or multiple responses on the left-hand side (see <doi: 10.18637/jss.v034.101>).”, Comprehensive R Archive Network (CRAN), 2020-10-16 13:50:06 UTC
Gbm, 2.1.8, “An implementation of extensions to Freund and Schapire's AdaBoost algorithm and Friedman's gradient boosting machine. Includes regression methods for least squares, absolute loss, t-distribution loss, quantile regression, logistic, multinomial logistic, Poisson, Cox proportional hazards partial likelihood, AdaBoost exponential loss, Huberized hinge loss, and Learning to Rank measures (LambdaMart). Originally developed by Greg Ridgeway.”, Comprehensive R Archive Network (CRAN), 2020-07-15 10:00:02 UTC
GGally, 2.1.0, “The R package ‘ggplot2’ is a plotting system based on the grammar of graphics. ‘GGally’ extends ‘ggplot2’ by adding several functions to reduce the complexity of combining geometric objects with transformed data. Some of these functions include a pairwise plot matrix, a two group pairwise plot matrix, a parallel coordinates plot, a survival plot, and several functions to plot networks.”, Comprehensive R Archive Network (CRAN), 2021-01-06 01:20:06 UTC
Ggnewscale, 0.4.5, Use multiple fill and colour scales in ‘ggplot2’, Comprehensive R Archive Network (CRAN), 2021-01-11 08:00:35 UTC
ggplot2, 3.3.5 “A system for ‘declaratively’ creating graphics, based on ““The Grammar of Graphics””. You provide the data, tell ‘ggplot2’ how to map variables to aesthetics, what graphical primitives to use, and it takes care of the details.”, Comprehensive R Archive Network (CRAN), 2021-06-25 11:30:09 UTC
ggpubr0.4.0, “The ‘ggplot2’ package is excellent and flexible for elegant data visualization in R. However the default generated plots requires some formatting before we can send them for publication. Furthermore, to customize a ‘ggplot’, the syntax is opaque and this raises the level of difficulty for researchers with no advanced R programming skills. ‘ggpubr’ provides some easy-to-use functions for creating and customizing ‘ggplot2’-based publication ready plots.”, Comprehensive R Archive Network (CRAN), 2020-06-27 06:20:02 UTC
ggrepel, 0.9.1, “Provides text and label geoms for ‘ggplot2’ that help to avoid overlapping text labels. Labels repel away from each other and away from the data points.”, Comprehensive R Archive Network (CRAN), 2021-01-15 22:00:02 UTC
glmnet, 4.1, Extremely efficient procedures for fitting the entire lasso or elastic-net regularization path for linear regression, logistic and multinomial regression models, Poisson regression, Cox model, multiple-response Gaussian, and the grouped multinomial regression. There are two new and important additions. The family argument can be a GLM family object, which opens the door to any programmed family. This comes with a modest computational cost, so when the built-in families suffice, they should be used instead. The other novelty is the relax option, which refits each of the active sets in the path unpenalized. The algorithm uses cyclical coordinate descent in a path-wise fashion, as described in the papers listed in the URL below, Comprehensive R Archive Network (CRAN), 2021-01-11 08:00:30 UTC
gmodels, 2.18.1, Various R programming tools for model fitting, Comprehensive R Archive Network (CRAN), 2018-06-25 16:23:05 UTC
gridExtra, 2.3, “Provides a number of user-level functions to work with “grid” graphics, notably to arrange multiple grid-based plots on a page, and draw tables.”, Comprehensive R Archive Network (CRAN), 2017-09-09 14:12:08 UTC
Hmisc, 4.4-2, “Contains many functions useful for data analysis, high-level graphics, utility operations, functions for computing sample size and power, importing and annotating datasets, imputing missing values, advanced table making, variable clustering, character string manipulation, conversion of R objects to LaTeX and html code, and recoding variables.”, Comprehensive R Archive Network (CRAN), 2020-11-29 06:00:16 UTC
Imager, 0.42.3, “Fast image processing for images in up to 4 dimensions (two spatial dimensions, one time/depth dimension, one colour dimension). Provides most traditional image processing tools (filtering, morphology, transformations, etc.) as well as various functions for easily analysing image data using R. The package wraps ‘CImg’, <http://cimg.eu>, a simple, modern C++ library for image processing.”, Comprehensive R Archive Network (CRAN), 2020-05-11 16:40:06 UTC
Jpeg, 0.1-8.1, This package provides an easy and simple way to read, write and display bitmap images stored in the JPEG format. It can read and write both files and in-memory raw vectors., Comprehensive R Archive Network (CRAN), 2019-10-24 14:51:52 UTC
Knitr, 1.31, “Provides a general-purpose tool for dynamic report generation in R using Literate Programming techniques.”, Comprehensive R Archive Network (CRAN), 2021-01-27 15:10:05 UTC
Lattice, 0.20-41, “A powerful and elegant high-level data visualization system inspired by Trellis graphics, with an emphasis on multivariate data. Lattice is sufficient for typical graphics needs, and is also flexible enough to handle most nonstandard requirements. See ? Lattice for an introduction.”, Comprehensive R Archive Network (CRAN), 2020-04-02 12:00:06 UTC
Libcoin, 1.0-8, “Basic infrastructure for linear test statistics and permutation inference in the framework of Strasser and Weber (1999) <https://epub.wu.ac.at/102/>. This package must not be used by end-users. Comprehensive R Archive Network (CRAN) package ‘coin’ implements all user interfaces and is ready to be used by anyone.”, Comprehensive R Archive Network (CRAN), 2021-02-08 11:00:03 UTC
Magrittr, 2.0.1, “Provides a mechanism for chaining commands with a new forward-pipe operator, %>%. This operator will forward a value, or the result of an expression, into the next function call/expression. There is flexible support for the type of right-hand side expressions. For more information, see package vignette. To quote Rene Magritte, “Ceci n'est pas un pipe.””, Comprehensive R Archive Network (CRAN), 2020-11-17 16:20:06 UTC
Matrix, 1.3-2, “A rich hierarchy of matrix classes, including triangular, symmetric, and diagonal matrices, both dense and sparse and with pattern, logical and numeric entries. Numerous methods for and operations on these matrices, using ‘LAPACK’ and ‘SuiteSparse’ libraries.”, Comprehensive R Archive Network (CRAN), 2021-01-06 17:40:19 UTC
Mboost, 2.9-4, “Functional gradient descent algorithm (boosting) for optimizing general risk functions utilizing component-wise (penalised) least squares estimates or regression trees as base-learners for fitting generalized linear, additive and interaction models to potentially high-dimensional data. Models and algorithms are described in \doi{10.1214/07-STS242}, a hands-on tutorial is available from \doi{10.1007/s00180-012-0382-5}. The package allows user-specified loss functions and base-learners.”, Comprehensive R Archive Network (CRAN), 2020-12-10 09:40:02 UTC
MLeval, 0.3, Straightforward and detailed evaluation of machine learning models. ‘MLeval’ can produce receiver operating characteristic (ROC) curves, precision-recall (PR) curves, calibration curves, and PR gain curves. ‘MLeval’ accepts a data frame of class probabilities and ground truth labels, or, it can automatically interpret the Caret train function results from repeated cross validation, then select the best model and analyse the results, ‘MLeval’ produces a range of evaluation metrics with confidence intervals, Comprehensive R Archive Network (CRAN), 2020-02-12 06:40:02 UTC
Mlogit, 1.1-1, “Maximum likelihood estimation of random utility discrete choice models. The software is described in Croissant (2020) <doi: 10.18637/jss.v095.i11> and the underlying methods in Train (2009) <doi: 10.1017/CBO9780511805271>.”, Comprehensive R Archive Network (CRAN), 2020-10-02 12:12:05 UTC
Modeltools, 0.2-23, “A collection of tools to deal with statistical models. The functionality is experimental and the user interface is likely to change in the future. The documentation is rather terse, but packages ‘coin’ and ‘party’ have some working examples. However, if you find the implemented ideas interesting we would be very interested in a discussion of this proposal. Contributions are more than welcome!”, Comprehensive R Archive Network (CRAN), 2020-03-05 11:50:06 UTC
Mvtnorm, 1.1-1, “Computes multivariate normal and t probabilities, quantiles, random deviates and densities.”, Comprehensive R Archive Network (CRAN), 2020-06-09 15:50:02 UTC
NCmisc, 1.1.6, “A set of handy functions, Includes a versatile one line progress bar, one line function timer with detailed output, time delay function, text histogram, object preview, Comprehensive R Archive Network (CRAN) package search, simpler package installer, Linux command install check, a flexible Mode function, top function, simulation of correlated data, and more.”, Comprehensive R Archive Network (CRAN), 2018-11-12 00:00:03 UTC
Party, 1.3-6, “A computational toolbox for recursive partitioning. The core of the package is ctree( ), an implementation of conditional inference trees which embed tree-structured regression models into a well defined theory of conditional inference procedures. This non-parametric class of regression trees is applicable to all kinds of regression problems, including nominal, ordinal, numeric, censored as well as multivariate response variables and arbitrary measurement scales of the covariates. Based on conditional inference trees, cforest( ) provides an implementation of Breiman's random forests. The function mob( ) implements an algorithm for recursive partitioning based on parametric models (e.g. linear models, GLMs or survival regression) employing parameter instability tests for split selection. Extensible functionality for visualizing tree-structured regression models is available. The methods are described in Hothorn et al. (2006) <doi: 10.1198/106186006X133933>, Zeileis et al. (2008) <doi: 10.1198/106186008X319331> and Strobl et al. (2007) <doi: 10.1186/1471-2105-8-25>.”, Comprehensive R Archive Network (CRAN), 2021-02-08 12:20:02 UTC
Partykit, 1.2-12, “A toolkit with infrastructure for representing, summarizing, and visualizing tree-structured regression and classification models. This unified infrastructure can be used for reading/coercing tree models from different sources (‘rpart’, ‘RWeka’, ‘PMML’) yielding objects that share functionality for print( )/plot( )/predict( ) methods. Furthermore, new and improved reimplementations of conditional inference trees (ctree( )) and model-based recursive partitioning (mob( )) from the ‘party’ package are provided based on the new infrastructure. A description of this package was published by Hothorn and Zeileis (2015) <https://jmlr.org/papers/v16/hothorn15a.html>.”, Comprehensive R Archive Network (CRAN), 2021-02-08 15:10:03 UTC
PerformanceAnalytics, 2.0.4, “Collection of econometric functions for performance and risk analysis. In addition to standard risk and performance metrics, this package aims to aid practitioners and researchers in utilizing the latest research in analysis of non-normal return streams. In general, it is most tested on return (rather than price) data on a regular scale, but most functions will work with irregular return data as well, and increasing numbers of functions will work with P&L or price data where possible.”, Comprehensive R Archive Network (CRAN), 2020-02-06 12:10:11 UTC
plotROC, 2.2.1, “Most ROC curve plots obscure the cutoff values and inhibit interpretation and comparison of multiple curves. This attempts to address those shortcomings by providing plotting and interactive tools. Functions are provided to generate an interactive ROC curve plot for web use, and print versions. A Shiny application implementing the functions is also included.”, Comprehensive R Archive Network (CRAN), 2018-06-23 07:52:56 UTC
plyr, 1.8.6, “A set of tools that solves a common set of problems: you need to break a big problem down into manageable pieces, operate on each piece and then put all the pieces back together. For example, you might want to fit a model to each spatial location or time point in your study, summarise data by panels or collapse high-dimensional arrays to simpler summary statistics. The development of ‘plyr’ has been generously supported by ‘Becton Dickinson’.”, Comprehensive R Archive Network (CRAN), 2020-03-03 14:40:02 UTC
png, 0.1-7, This package provides an easy and simple way to read, write and display bitmap images stored in the PNG format. It can read and write both files and in-memory raw vectors., Comprehensive R Archive Network (CRAN), 2013-12-03 22:25:05
pROC, 1.17.0.1, Tools for visualizing, smoothing and comparing receiver operating characteristic (ROC curves). (Partial) area under the curve (AUC) can be compared with statistical tests based on U-statistics or bootstrap. Confidence intervals can be computed for (p)AUC or ROC curves, Comprehensive R Archive Network (CRAN), 2021-01-13 14:30:02 UTC
psych, 2.0.12, A general purpose toolbox for personality, psychometric theory and experimental psychology. Functions are primarily for multivariate analysis and scale construction using factor analysis, principal component analysis, cluster analysis and reliability analysis, although others provide basic descriptive statistics. Item Response Theory is done using factor analysis of tetrachoric and polychoric correlations. Functions for analyzing data at multiple levels include within and between group statistics, including correlations and factor analysis. Functions for simulating and testing particular item and test structures are included. Several functions serve as a useful front end for structural equation modeling. Graphical displays of path diagrams, factor analysis and structural equation models are created using basic graphics. Some of the functions are written to support a book on psychometric theory as well as publications in personality research. For more information, see the <https://personality-project.org/r/> web page, Comprehensive R Archive Network (CRAN), 2020-12-16 16:10:03 UTC
readxl, 1.3.1, “Import excel files into R. Supports ‘.xls’ via the embedded ‘libxls’ C library <https://github.com/libxls/libxls> and ‘.xlsx’ via the embedded ‘RapidXML’ C++ library <http://rapidxml.sourceforge.net>. Works on Windows, Mac and Linux without external dependencies.”, Comprehensive R Archive Network (CRAN), 2019-03-13 16:30:02 UTC
reshape2, 1.4.4, “Flexibly restructure and aggregate data using just two functions: melt and ‘dcast’ (or ‘acast’).”, Comprehensive R Archive Network (CRAN), 2020-04-09 13:50:02 UTC
ROCR, 1.0-11, “ROC graphs, sensitivity/specificity curves, lift charts, and precision/recall plots are popular examples of trade-off visualizations for specific pairs of performance measures. ROCR is a flexible tool for creating cutoff-parameterized 2D performance curves by freely combining two from over 25 performance measures (new performance measures can be added using a standard interface). Curves from different cross-validation or bootstrapping runs can be averaged by different methods, and standard deviations, standard errors or box plots can be used to visualize the variability across the runs. The parameterization can be visualized by printing cutoff values at the corresponding curve positions, or by coloring the curve according to cutoff. All components of a performance plot can be quickly adjusted using a flexible parameter dispatching mechanism. Despite its flexibility, ROCR is easy to use, with only three commands and reasonable default values for all optional parameters.”, Comprehensive R Archive Network (CRAN), 2020-05-02 14:50:05 UTC
Rpart, 4.1-15, “Recursive partitioning for classification, regression and survival trees. An implementation of most of the functionality of the 1984 book by Breiman, Friedman, Olshen and Stone.”, Comprehensive R Archive Network (CRAN), 2019-04-12 14:32:39 UTC Sandwich, 3.0-0, “Object-oriented software for model-robust covariance matrix estimators. Starting out from the basic robust Eicker-Huber-White sandwich covariance methods include: heteroscedasticity-consistent (HC) covariances for cross-section data; heteroscedasticity-and autocorrelation-consistent (HAC) covariances for time series data (such as Andrews' kernel HAC, Newey-West, and WEAVE estimators); clustered covariances (one-way and multi-way); panel and panel-corrected covariances; outer-product-of-gradients covariances; and (clustered) bootstrap covariances. All methods are applicable to (generalized) linear model objects fitted by Im( ) and glm( ) but can also be adapted to other classes through S3 methods. Details can be found in Zeileis et al. (2020) <doi: 10.18637/jss.v095.i01>, Zeileis (2004) <doi: 10.18637/jss.v011.110> and Zeileis (2006) <doi: 10.18637/jss.v016.109>.”, Comprehensive R Archive Network (CRAN), 2020-10-02 10:40:02 UTC
Stabs, 0.6-4, “Resampling procedures to assess the stability of selected variables with additional finite sample error control for high-dimensional variable selection procedures such as Lasso or boosting. Both, standard stability selection (Meinshausen & Buhlmann, 2010, <doi: 10.1111/j.1467-9868.2010.00740.x>) and complementary pairs stability selection with improved error bounds (Shah & Samworth, 2013, <doi: 10.1111/j.1467-9868.2011.01034.x>) are implemented. The package can be combined with arbitrary user specified variable selection approaches.”, Comprehensive R Archive Network (CRAN), 2021-01-29 10:00:02 UTC
Strucchange, 1.5-2, “Testing, monitoring and dating structural changes in (linear) regression models. strucchange features tests/methods from the generalized fluctuation test framework as well as from the F test (Chow test) framework. This includes methods to fit, plot and test fluctuation processes (e.g., CUSUM, MOSUM, recursive/moving estimates) and F statistics, respectively. It is possible to monitor incoming data online using fluctuation processes. Finally, the breakpoints in regression models with structural changes can be estimated together with confidence intervals. Emphasis is always given to methods for visualizing the data.”, Comprehensive R Archive Network (CRAN), 2019-10-12 18:35:49 UTC
Survival, 3.2-7, “Contains the core survival analysis routines, including definition of Surv objects, Kaplan-Meier and Aalen-Johansen (multi-state) curves, Cox models, and parametric accelerated failure time models.”, Comprehensive R Archive Network (CRAN), 2020-09-28 08:20:02 UTC
VIM, 6.1.0, “New tools for the visualization of missing and/or imputed values are introduced, which can be used for exploring the data and the structure of the missing and/or imputed values. Depending on this structure of the missing values, the corresponding methods may help to identify the mechanism generating the missing values and allows to explore the data including missing values. In addition, the quality of imputation can be visually explored using various univariate, bivariate, multiple and multivariate plot methods. A graphical user interface available in the separate package VIMGUI allows an easy handling of the implemented plot methods.”, Comprehensive R Archive Network (CRAN), 2021-01-19 17:00:13 UTC
Xlsx, 0.6.5, Provide R functions to read/write/format Excel 2007 and Excel 97/2000/XP/2003 file formats, Comprehensive R Archive Network (CRAN), 2020-11-10 15:00:02 UTC
Xts, 0.12.1, Provide for uniform handling of R's different time-based data classes by extending zoo, maximizing native format information preservation and allowing for user level customization and extension, while simplifying cross-class interoperability, Comprehensive R Archive Network (CRAN), 2020-09-09 16:40:03 UTC
Zoo, 1.8-8, “An S3 class with methods for totally ordered indexed observations. It is particularly aimed at irregular time series of numeric vectors/matrices and factors. zoo's key design goals are independence of a particular index/date/time class and consistency with ts and base R by providing methods to extend standard generics.”, Comprehensive R Archive Network (CRAN), 2020-05-02 16:14:00 UTC

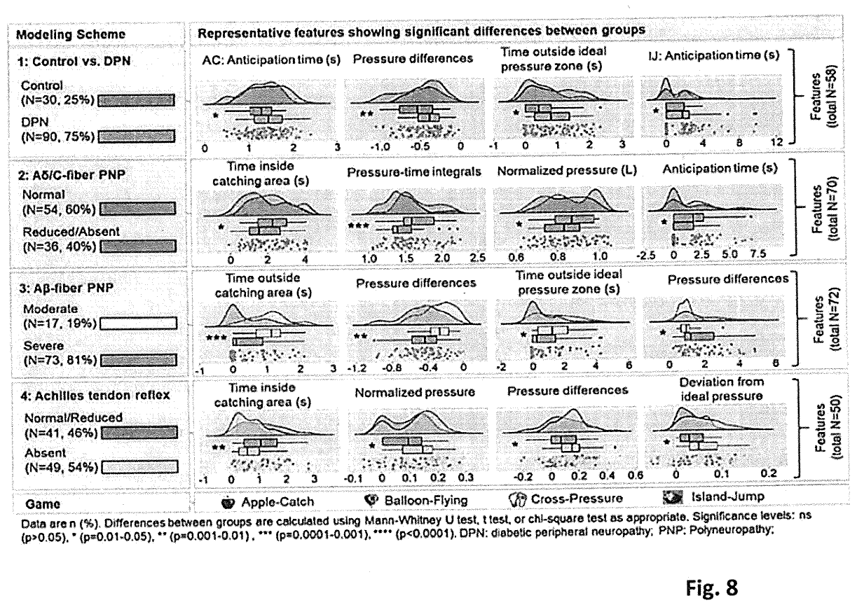

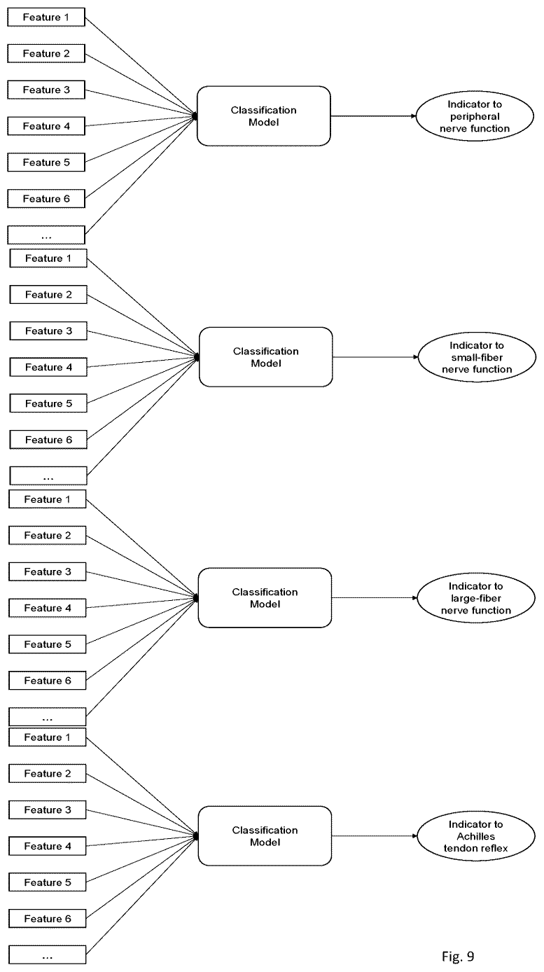

FIG. 9 schematically shows, how the computer implemented method, as the artificial intelligence model, functions.

As presented in the FIG. 9, the inputs can be extracted features/parameters from sensor data and game results. Output can be a predictive indicator of possibility that the person suffers from impairment of differently sized nerves. The classification model can save the data patterns of healthy subjects and individuals with peripheral neuropathy, which are learnt from previous acquired datasets. The workflow can be that the model tries to draw a conclusion on the possibility that the person suffers from peripheral neuropathy from inputted values. This calculation process can happens in the data process device 14. The applied models to learn the data patterns can be support vector machine (SVM), random forest (RF), artificial neural network (ANN), etc.

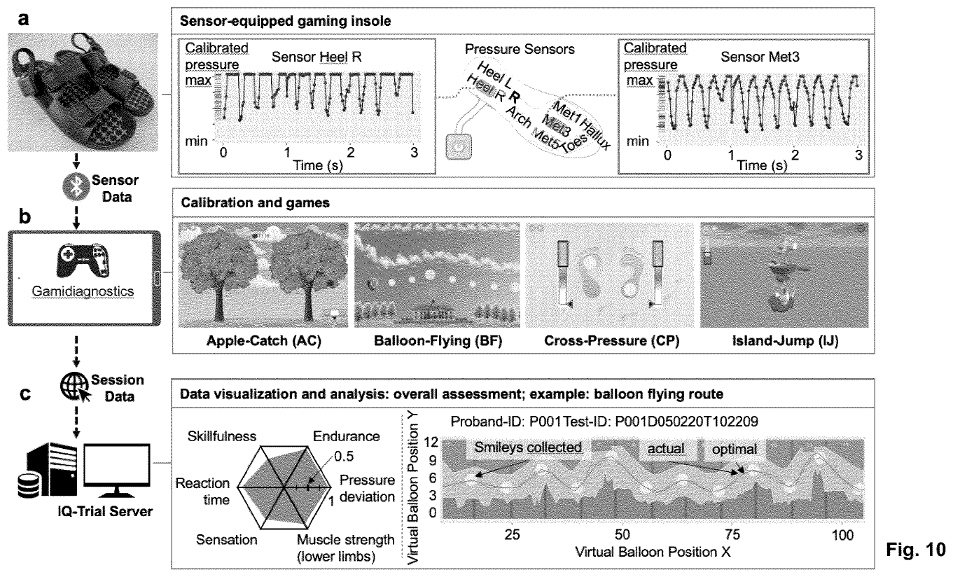

FIG. 10 an overview of an exemplary embodiment of the system 10. Field A contains shoes with sensor-equipped gaming insoles harboring eight embedded pressure sensors 20 (not depicted in FIG. 10) in distinct areas of the plantar pedis. Top center image of FIG. 10 shows localization of sensors in the heel at the calcaneus, arch, metatarsal (Met) 1/3/5, toes at the forefoot, and hallux at digitus. The insole serves as a steering unit and is connected to a control unit for real-time data transmission, for example wirelessly, e.g. by via Bluetooth, to the game application, which is run on a tablet shown in field B. The setup allows the person to play games solely by modulating plantar pressure values. Each gaming session may include eight calibration steps and four games that is Balloon-Flying (BF), Apple-Catch (AC), Cross-Pressure (CP), and Island-Jump (IJ), as shown in the middle of the center image. Subsequently, the data may be uploaded to a server shown in field C as a data package. The persons may receive a short summary of their performance with a spider chart, scores, levels, and capabilities (beginner, average, advanced, expert), as shown at the bottom of the center image. Further data visualization for the physician and data analyses may be realized to indicate the performance of persons in the different games in comparison to maximum achievement levels.

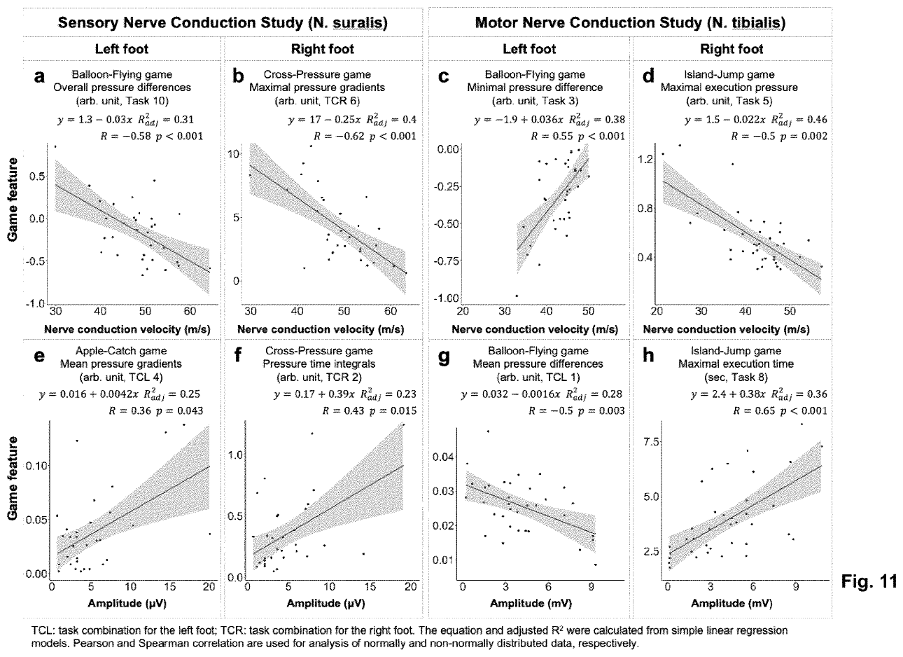

FIG. 11 shows exemplary correlations between nerve conduction velocity and features derived from the sensor data gathered by the person playing the respective games. TCL means “task combination for the left foot”; TCR means “task combination for the right foot”. The equation and adjusted R² were calculated from simple linear regression models. Pearson and Spearman correlation were used for the analysis of normally and non-normally distributed data, respectively.

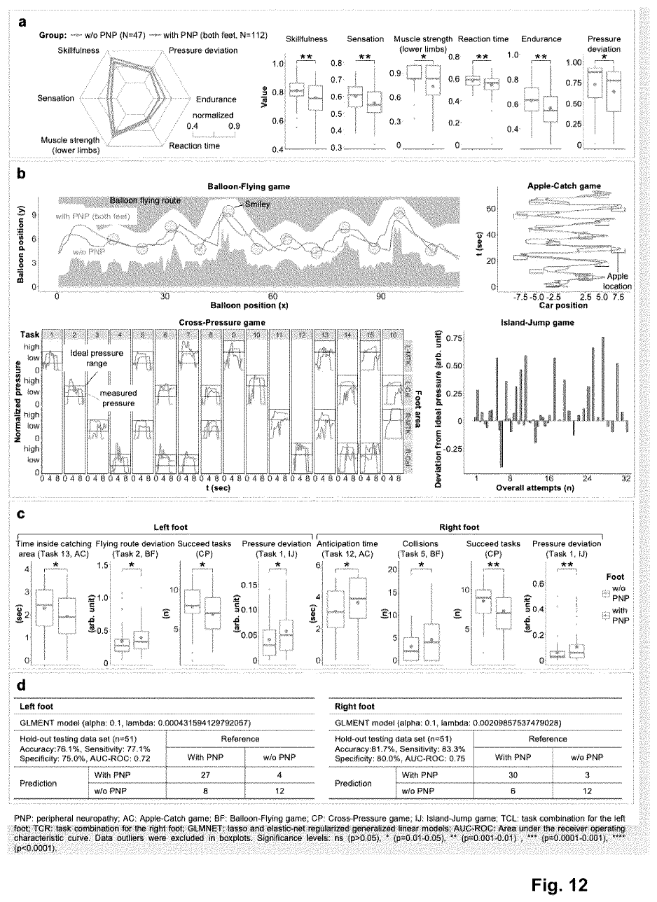

In FIG. 12, a peripheral neuropathy classification and game-based findings in a subcohort are shown. Presentation of hypothesis-driven key capabilities between persons without (w/o) and with symmetrical PNP are shown in field A. Visualization of game performance of two studied persons (healthy individual without PNP, patient with symmetrical PNP) are shown in field B. Representative game features correlated with the presence of PNP are shown in field C. Predictive performance evaluation of trained classification models to discriminate feet with PNP from healthy feet (w/o PNP) are shown in field D. Abbreviations used in FIG. 12 are: PNP: peripheral neuropathy; AC: Apple-Catch game; BF: Balloon-Flying game; CP: Cross-Pressure game; IJ: Island-Jump game; TCL: task combination for the left foot; TCR: task combination for the right foot; GLMNET: lasso and elasticnet regularized generalized linear models; AUC-ROC: Area under the receiver operating characteristic curve.

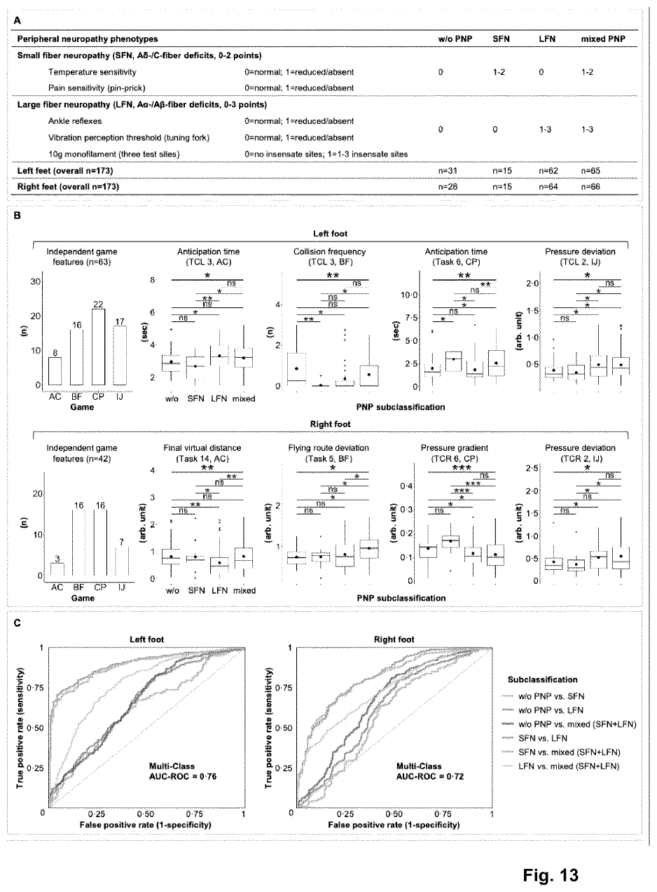

FIG. 13 depicts nerve damage pattern according to clinical examination and correlation with game-based findings. Field A shows clinical examination results of small and large fiber neuropathy. Field B shows game features that correlate significantly with nerve fiber damage subclassification. Field C is a visualization of multi-class AUC-ROC curves for the PNP subclassification (small/large/mixed nerve fiber dysfunctions). Abbreviations used in FIG. 13 are: PNP: peripheral neuropathy; SFN: small fiber neuropathy; LFN: large fiber neuropathy; AC: Apple-Catch game; BF: Balloon-Flying game; CP: Cross-Pressure game; IJ: Island-Jump game; TCL: task combination for the left foot; TCR: task combination for the right foot; AUC-ROC: Area under the receiver operating characteristic curve.

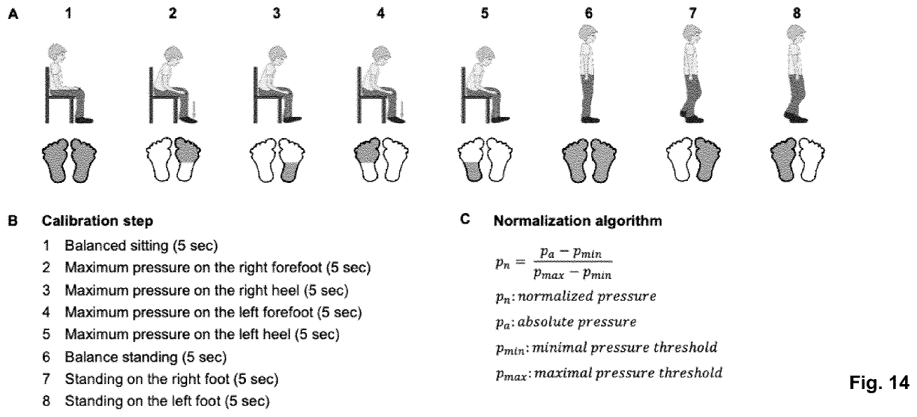

FIG. 14 shows an exemplary embodiment of a calibration routine for calibrating the foot pressure sensors. Exemplary actions to be performed for the calibration routine by the person are depicted in section A of FIG. 14. The exemplary calibration steps of section A are detailed in section B of FIG. 14. An applicable normalization algorithm is shown in section C.

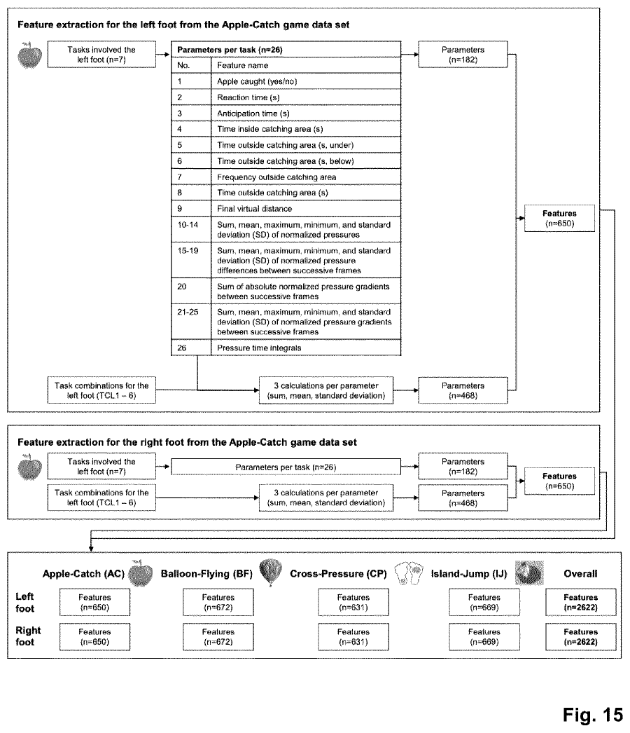

FIG. 15 shows an exemplary embodiment of how to extract features for the PNP classification model for game feature analysis of a subcohort. NCS were performed in 37 patients (subcohort 1), all diagnosed with diabetes and normal cognitive status (MoCA=26). This subcohort had similar distributions in age, weight, BMI, and neuropathic scores as the entire cohort. The gender distribution was lightly skewed towards female patients in the subcohort. Patients' game performance was significantly correlated to the sensory and motor conduction velocity (NCV) and amplitude in the lower extremities. Feature extraction of exemplary distinct parameters from four games was set up for each foot side. Overall, 277 independent exemplary game features revealed obvious associations with NCS parameters. Eight representative exemplary features are depicted in FIG. 11. A strongest correlation may be between the motor nerve conduction amplitude of the right foot and the maximal execution time in task 8 of the Island-Jump game (R=0.65, p<0.001, adjusted R2=0.36, section H of FIG. 11). BF and AC games may contribute further features that may be correlated with the NCV and amplitude of the left foot. However, more features extracted from BF and IJ games may be associated with the NCV and amplitude of the right foot. 164 and 113 independent exemplary game features seem to significantly correlate with motor and sensory nerve states, respectively. 146 independent exemplary game features seem to be associated with NCV, and 131 seem to correlate to the amplitude of nerves of the foot.

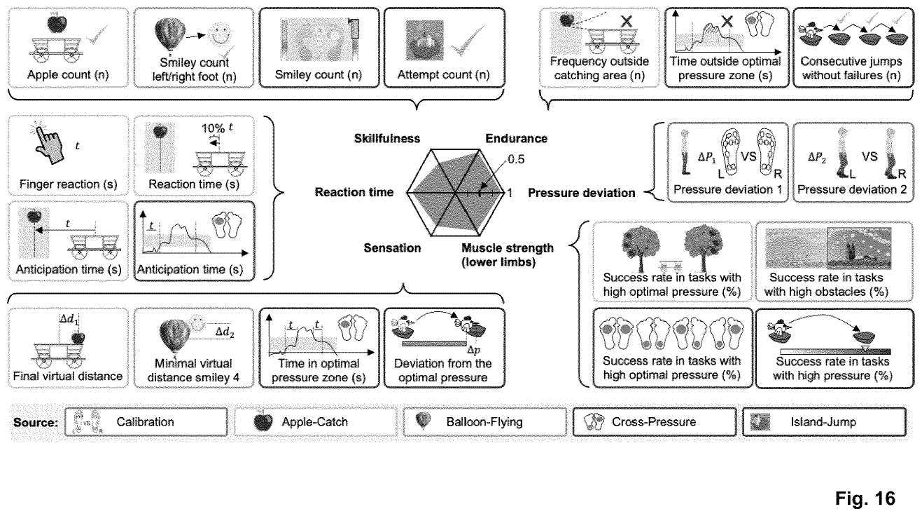

FIG. 16 represents an exemplary embodiment of considerations on key capabilities and scoring of virtual games. For example, out of all persons (e.g. n=329), a subcohort 2 with normal cognitive state (MoCA≥26) aged >55 years may be generated. No significant differences in age, gender, and BMI were between patients without (w/o) and with clinical PNP. Individuals with neuropathy had a significantly longer duration of diabetes and more severe neuropathic symptoms. Clinically evidence for PNP was present in 118 left and 120 right feet. For all these feet, at least one insensate site during the 10-g monofilament test were found. PNP was identified in both feet in 112 patients. No PNP was present in 47 patients, 26% of patients had unilateral PNP.

According to the invention, critical skills relate to peripheral nerve functions and are useful for successful game performance. As an example, six key capabilities are defined: reaction time (understanding; immediate response to tasks), sensation (fine-tuning of pressure application in subtasks), skillfulness (overall achievements in each game), muscle strength (lower limbs) (achievements with high-pressure application), plantar pressure deviation (pressure distribution left versus right foot) and endurance (steadiness of pressure application in tasks), as depicted in FIG. 16. Selected or all of the key capabilities may reveal significant differences between patients without PNP (n=47) and with symmetrical PNP (n=112). Exemplary game performance differences between two persons are visualized for all games (w/o PNP; with PNP; FIG. 12 section B). Out of 2,622 extracted game features, for example 50 and 56 independent game features may be useful to reveal differences between the “w/o PNP” and “with PNP” groups. Representative examples of these game features were compared and visualized for each game (FIG. 12 section C).

With the extracted game features from 173 patients in subcohort 2, predictive models for PNP were trained exemplarily using the five-fold ten repeats cross-validation. For example 70% of game data sets were utilized for model training and 30% for testing. In the hold-out testing data set, the obtained Lasso and Elastic-Net Regularized Generalized Linear Model (GLMNET) identified PNP in the left foot with an adjusted accuracy of 76.1% (sensitivity 77.1%, specificity 75.0%, AUC-ROC 0.72, FIG. 12 section D). For determining PNP in the right foot, another GLMNET model achieved an accuracy of 81.7% (sensitivity 83.3%, specificity 80.0%, AUC-ROC 0.75, FIG. 12 section D).

In addition, for example, multi-classification models were established to differentiate feet without PNP (w/o) versus SFN, LFN, and mixed nerve fiber damages. Exemplary game features were extracted separately for the left and right foot from the game data set. The trained Gradient Boosting Models (GBM) yielded a multi-class AUC of 0.76 and 0.72 when identifying the nerve fiber damage pattern for the left and right foot, respectively (cf. FIG. 13 section C). Both models performed better in distinguishing between SFN and LFN.

### REFERENCE SIGNS

1 method
2 start
3 bring feet in contact with sensors
4 play game and gather data
5 analyze sensor data and indicate or determine peripheral neuropathy
6 end
10 system
11,12 foot bottom pressure sensors
14 data processing device
20 pressure sensitive element
21 signal to data converter
22 data interface

1. A computer implemented method for indicating, whether or not a person suffers from peripheral neuropathy,
characterized in that the method comprises
loading sensor data from foot bottom pressure sensors that were pressed between the feet of the person and the underground while the person was playing a predetermined computer game while controlling the computer game by exerting pressure on the pressure sensors via the person's feet, and
analyzing the sensor data with an algorithm that is adapted to detect whether or not the person suffers from peripheral neuropathy based on the sensor data.

2. The method of claim 1, characterized in that the sensor data and game data concerning the person's gaming performance are combined and analyzed by the algorithm.

3. The method of claim 1, characterized in that the sensor data include data representing the amount of pressure exerted by the person and/or the timing of the pressure exerted by the person.

4. The method of claim 2, characterized in that the game data include timing and/or accuracy of control of positioning an item of the game with respect to another item of the game.

5. The method of claim 1, characterized in that the algorithm is adapted to indicate the person's reaction time, anticipation time, sensation, skillfulness, endurance, plantar pressure deviation, and/or muscle strength of lower limbs from the sensor data or the combination of sensor data and game data.

6. The method of claim 1, characterized in that the algorithm is an artificial intelligence model trained to indicate whether a person suffered from peripheral neuropathy based at least on the pressure data.

7. A data processing device with a storage device and a processor, wherein the storage device comprises instruction, which, when executed by the processor cause the processor to carry out the method of claim 1.

8. A computer program comprising instructions, which, when the program is executed by a computer, cause the computer to carry out the method of claim 1.

9. A system for indicating, whether or not a person suffers from peripheral neuropathy,
characterized in that
the system comprises a gaming device for playing computer games,
at least two foot bottom pressure sensors that are connectable to the gaming device in a control signal transmitting manner, and
the data processing device of claim 7, wherein
the data processing device is adapted to load the sensor data from foot bottom pressure sensors, from the gaming device or from another storage device of the system.
